(19) United States
(12) Patent Application Publication
(10) Pub. No.: US 2018/0161183 A1
(43) Pub. Date: Jun. 14, 2018

(54) SYSTEM FOR CONNECTING A PROSTHESIS COMPONENT TO A PROSTHESIS SHAFT

(71) Applicant: Bernhard SACHERER, Friesenheim (DE)

(72) Inventor: Bernhard SACHERER, Friesenheim (DE)

(21) Appl. No.: 15/378,882

(22) Filed: Dec. 14, 2016

(51) Int. Cl. A61F 2/80; A61F 2/50

(52) U.S. Cl. CPC A61F 2/80; A61F 2002/607; A61F 2/5046

(57) ABSTRACT

A shaft system for a leg prosthesis with the shaft system featuring a prosthesis shaft which has a wall made of fiber-reinforced plastic material and which features at its proximal end an entrance opening and at its distal end a fastening assembly for the connection of prosthesis components and the attachment of tensioning elements and functional parts of the prosthesis such as locks, draw-in and connecting elements. In order to enable on such a shaft system the feasibility and faster adaptability to the requirements of a user of this shaft system, it is proposed to provide the aforementioned passage opening with at least one reinforcement element. This allows easier fitting of a prosthesis because of the possibility of a faster and simpler repositioning if needed or the faster exchange of prosthesis components.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

### 1. Field of the Invention

The invention concerns a shaft system for a leg prosthesis with the shaft system featuring a prosthesis shaft which has a wall made of fiber-reinforced plastic material and which features at its proximal end an entrance opening and at its distal end a fastening device for the connection of prosthesis components and for the fitting of tensioning de-vices and functional prosthesis parts such as locks, draw-in and connecting elements as well as tensioning devices.

Furthermore, the invention concerns an appropriate method for the manufacture of such a shaft system.

### 2. Description of Related Art Including Information Disclosed Under 37 CFR 1.97 and 37 CFR 1.98

It is known, for example from DE 10 2011 117 802 A1, that prostheses have a prosthesis shaft which is fitted to a body part, such as for in-stance an arm or leg stump, with the prosthesis shaft featuring a wall essentially surrounding the body part and consisting of thermoplastic or fiber-reinforced plastic material, with this prosthesis shaft, in particular at its distal end, being provided with fastening device for a prosthesis-connecting element by which artificial limbs such as hands, knee links or feet are attached to the prosthesis shaft.

The aforementioned fastening device at the distal end of the prosthesis shaft is frequently cast firmly and inseparably as a non-detachable adapter plate, for instance in the form of a four-hole-connecting plate or in the form of a threaded receptacle M36 etc. in the vicinity of the distal end of the prosthesis shaft. With the fastening device thus firmly connected to the prosthesis shaft, the static structure of the prosthesis, i.e. the relative position of the prosthesis shaft to the prosthesis components, has also been established.

To ensure its protection, the amputation stump is very frequently covered with a silicon liner whereby it is also possible to obtain an improved connection between the stump and the shaft of the prosthesis. For that last purpose, this so-called liner is provided for example with sealing lips or various connection elements, such as for instance a pin or draw-in ribbon, which are secured in a so-called lock at the distal end of the prosthesis shaft. This blocking device (lock) is executed in very different variants and, in a conventional prosthesis, is likewise solidly cast at the distal end of the prosthesis shaft or screwed into an adapter plate that has been cast at the distal end.

During the fitting of a leg prosthesis it is then sensible to give a user the opportunity to test several prosthesis connection elements such as insertion elements, locks or valves. For this, it is usually necessary to make modifications at the distal end of the prosthesis shaft. The most appropriate prosthesis connecting element and the static structure to be determined during the fitting are extremely important for the comfortable wear, manipulation and quality of the prosthesis.

In this context, it often happens that alterations, particularly in the area of the distal end of the prosthesis shaft, are required that are so costly that frequently the manufacture of a new prosthesis shaft is being considered instead.

The prosthesis connecting elements which are being integrated into the prosthesis shaft such as four-hole connecting plates or lock housings, as are also shown for example in US 2005/0244220, have so far always been provided with undercuts into which the plastic material of the prosthesis shaft wall fits in such a positive manner that a repeated exchange, as may be necessary, of the prosthesis connecting elements is often not possible without damaging the prosthesis shaft or the prosthesis connecting element in the process. In the case of a prosthesis connecting element that is inseparable from the prosthesis shaft, an actually sensible readjustment of the prosthesis is often not carried out because the expenditure for the modifications on the distal shaft end of the prosthesis are considered to be too high.

Despite the very costly connection between prosthesis shaft and prosthesis components described here, it also happens frequently, particularly during the tryout period of a prosthesis, that a loosening or a fracture occurs at the connecting point of prosthesis shaft and prosthesis connecting element. The connection of prosthesis shaft and prosthesis connecting elements is therefore, especially with leg prostheses a weak point that can potentially become a source of high risk for the user.

In order to be able to try out the various prosthesis connecting elements and also to determine the static structure of a prosthesis with a still reasonable expense, all prosthesis connecting elements positioned at the distal end of the prosthesis shaft and functional parts should be installed in a completely exchangeable and modifiable manner without being obliged to cause any damage or modification on the prosthesis shaft proper during the exchange.

It is therefore the aim of the invention to make available a reliable connection of the prosthesis shaft and the prosthesis components which also permits a quick exchange of all prosthesis connecting elements and functional parts of the prosthesis without damaging the prosthesis shaft during an exchange.

A manufacturing method according to the invention shall thus ensure a reliable connection in the sense of optimal stability, precision and adjustability with an optically pleasing surface without any reworking on the prosthesis shaft.

### BRIEF SUMMARY OF THE INVENTION

According to the invention, this aim is achieved by the fastening device provided for the prosthesis connecting element in the wall of the prosthesis shaft and which features a passage opening for the fixation of a connection piece, with at least one reinforcing element preferably made of metal, synthetic or composite material being provided at this passage opening and which is connected with high stability to the synthetic material of the prosthetic shaft.

Such a passage opening has the particular advantage that by means of this passage opening prosthesis connecting parts can be fastened in detachable manner on the wall of the prosthesis shaft and can thus be twisted especially in their relative position to the prosthesis.

The primary function of this reinforcing element at the distal end of the prosthesis shaft consists in safely transmitting the acting forces of the prosthesis components such as knee link or prosthetic foot to the fiber-reinforced synthetic material of the shaft wall.

For this purpose, the reinforcement in an implementation, according to the invention, is embedded into the synthetic material around the passage opening and thereby profoundly and non-detachably connected with the fiber reinforcement of the shaft wall and can additionally be twisted between re-adjustable tensioning elements.

All prosthesis connecting elements are also detachably connected with the passage opening at the distal end of the prosthesis shaft and can be detached from it on demand and thus be significantly modified in their location or position.

The advantage of the invention lies in the fact that at the distal end of the prosthesis shaft a reinforcement element especially made of metal, synthetic or composite material can be connected with high stability to the synthetic material of the prosthetic shaft. In particular, such a rein-forcing element then permits a sufficient size of the passage opening for the introduction of a connecting piece into it, with such a connecting piece being usually located on prosthesis connecting elements and/or functional parts of the prosthesis.

In a particularly preferred implementation of the invention, the part of the prosthesis connecting element which runs through the passage opening, while rotatable and arrestable with respect to the axis of the limb, is firmly anchored at the passage opening in the wall of the prosthesis shaft.

As explained, there may also be provided in the passage opening especially a functional part of the prosthesis such as a lock, a draw-in and fastening element as well as also a clamping element for arresting a connection piece or similar, thereby optimizing especially the headroom to be provided for add-on pieces to the prosthesis.

During the manufacture of a prosthesis shaft made of fiber-reinforced synthetic material, it is customary to install the prosthesis connecting element for prosthesis components at the distal end of the shape model. The prosthesis connecting element is here frequently realized as a combined functional unit with mechanical connecting elements such as liner-lock and draw-in elements and additionally with a standardized four-hole receptacle for the connection of prosthesis add-on pieces and is made at least partly or entirely of metal.

For the manufacture of the shaft it is customary to attach a prosthesis connecting element on the distal end of a shape model of an amputation stump and is connected with a fiber reinforcement of the plastic material the prosthesis shaft is made of. This fiber reinforcement during the manufacture of the prosthesis is in the form of a fabric sleeve.

During the manufacture of the prosthesis stump an excess length of the fabric sleeve is kept over the full length of the shape model. In the area of the prosthesis-connecting element the fiber reinforcement has until now been laced tightly with a manually introduced lacing into a cavity at the circumference of the prosthesis connecting element. With this lacing the fabric sleeve is firmly connected to the prosthesis connecting element. The mentioned excess length of the fabric sleeve is then folded back over said lacing and is placed back over the shape model.

For an additional strengthening in the direction of the circumference of the prosthesis shaft, more fibers running in the direction of the circumference are then placed around the prosthesis connecting element, in particular in the form of specially fitting cuttings. These steps are repeated several times during the manufacture of a prosthesis shaft.

This is described in the publication of the Össur Company, Reykjavik, Iceland: Technical Manual Icelook 200 Series Lamination Procedure, Version 1, EB 345/1 JG March 2000, pp. 1-20.

One disadvantage of this manufacturing method is that the peripherally placed layers of fiber reinforcement and hence essential components of the shaft wall cannot directly engage into the cavity of the prosthesis connecting element. For that reason, the contact of the fiber-reinforced plastic of the shaft wall with the cavity on the prosthesis-connecting element exists only over the first layers of the fiber-reinforced plastic which are immediately placed on the prosthesis connecting element.

For the cast resin method usually applied for the manufacture of prostheses, the fiber material draped in the manner described above is en-closed with a vacuum hose and filled with a plastic matrix. This plastic matrix reacts as it is being heated. Considering that metal parts of the prosthesis-connecting element and the plastic material of the shaft wall situated at the circumference of the prosthesis-connecting element have different thermal expansion coefficients, temperature variations result inevitably in a relative motion and thus in an undesirable loosening of the connection among the various materials involved.

Heavy demands on the prosthesis-connecting element may also lead to delamination of the fiber-reinforced plastic and thus to a loosening of the connection of the prosthesis connecting element with the prosthesis shaft.

Thus, the invention is also based on the idea that an optimized connection of the fiber-reinforced plastic of the shaft wall with the prosthesis connecting element is ensured only when every single fiber of the fiber-reinforced plastic of the prosthesis shaft with its maximal mechanical load capacity is anchored on the prosthesis connecting element and can therefore be effective.

Since the fiber-reinforced plastic of a prosthesis shaft is in principle applied in individual layers or strata, it is therefore suggested to connect each of these layers directly with a reinforcing element that is integrated into the rim of the passage opening and thus to build up in layers a connection of such reinforcing elements with the prosthesis shaft.

An optimized connection of the prosthesis shaft with the reinforcing elements is thus ensured, according to the invention, by guiding each single layer of the fiber fabric through a circular lamella acting as a reinforcing element surrounding the passage opening at the distal end of the prosthesis shaft. Later these fibers will be protected by the plastic matrix surrounding them and they may also have a positive fit with the circular lamella. Regardless of the adhesive qualities of the combined materials, the positive locking of the enveloping fiber with the reinforcing element in the form of, for example, a circular lamella ensures a se-cure mechanical connection of the reinforcing elements with the fiber-reinforced plastic material.

In principle, the reinforcing elements lying in axial direction next to each other at the end may not only be in the form of circular lamellas, but it is also within the scope of the invention to realize these reinforcing elements as string loops. These may either be prefabricated or be bound individually. What matters is that they will eventually be essentially positioned one behind the other in axial direction and not in radial direction essentially one above the other.

It is in accordance with the invention that the rim of the passage opening can be held together with tensioning devices, which also counteracts any possible delamination. In case of a loosening, the connection can very simply and effectively be retightened by taking up the tensioning devices.

These tensioning devices may be provided additionally, when the rim of the passage opening, according to the invention, has been provided with integrated reinforcing elements, for the purpose of further strengthening the strong bond that is present here between fibers and circular lamellas or similar.

These tensioning devices may consist for example of a central tensioning or clamping device passing through the passage opening or of screw type elements passing through bore holes inside the reinforcing elements.

Since the fiber orientation of the shaft wall is led through the opening in the center of the reinforcing elements, i.e. for example of a circular lamella or a string loop, and the reinforcing elements thereby become very intimately combined with the plastic material and the fiber reinforcement of the shaft wall, the effect of different thermal expansion coefficients of metal and plastic is used to advantage: As the circular measure of the reinforcing element, preferably consisting of a metallic circular lamella, expands during the manufacturing process and then shrinks during the cooling period, this results in the plastic matrix achieving a high-strength form-fit on this reinforcing element which is for example in the form of a circular lamella.

In this manner, a manufacturing method according to the invention, ensures constant quality of the connection between reinforcing elements and the prosthesis shaft.

During the manufacture of the passage opening a place-holder is installed, in lieu of a connection piece, at the distal top of the positive shape model. This serves first to keep open the location for the passage opening at the distal end of the shaft. This place-holder, as well as the shape model, are first sheathed, in a manner known as such, in a subfoil and then in a first layer of a reinforcing fiber woven as a hose for the synthetic material to be reinforced. Here, too, an overlapping excess over the entire length of the shape model is maintained.

It is in conformance with the invention that for the purpose of inverting the fiber fabric in lieu of a lacing it is now a reinforcing element, in particular a circular lamella or also a string loop that is placed over the place-holder. In a particularly preferred implementation said circular lamella is made of metal or a composite material containing metal and displays high solidity so that this element is suitable for absorbing the forces created by threaded inserts and/or tension elements.

The reinforcing element, especially in form of a circular lamella, thus envelops at least one layer of the fiber fabric hose of the shaft wall.

It is also in accordance with the invention to combine differently formed reinforcement elements depending on their usefulness.

In a preferred implementation a reinforcing element in the form of a circular lamella features at least one extension in radial direction which can be placed between the layers of fiber reinforcement against the contour of the shape model.

This allows such a circular lamella to be connected with the fiber-reinforced synthetic material over a larger surface. This also makes it possible to stabilize and modify the reinforcing element as may be required.

The contour of the clear opening on the circular lamella follows the exterior contour of the place-holder which has exactly the shape of the connection piece to be placed there later. Regardless of the shape of the place-holder, it is between the circular lamella and the place-holder that the fiber reinforcement for the plastic material is being positioned. The aforementioned excess of fiber fabric is now being folded back at the circular lamella and placed as a second layer over the shape model.

This process can be repeated as often as desired in identical manufacturing steps and with constant quality and sturdiness. Every additional layer increases the stability of the connection of fiber-reinforced plastic material with the reinforcing elements or circular lamellas respectively in a continuous and calculable manner.

The exterior contour of the fiber fabric constitutes an evenly formed surface also in the distal connection zone of the prosthesis shaft with the passage opening created there, which does not require any finishing work.

According to the invention, to end the procedure from distal, a molded part, for example in the form of a connection piece, is positioned above the place-holder protruding there and is attached, with a proximally oriented shaped surface, to the fiber reinforcement wrapped around the reinforcing element or circular lamellas respectively. Finally, over this molded part and over the draped fiber fabric, a vacuum hose is placed which is being filled with the plastic matrix. Simultaneously and automatically the contact surface of the molding stamps a smooth bearing surface, for example at a right angle to the stump axis, over which pros-thesis-connecting pieces can be attached precisely on the wall off the prosthesis shaft.

In this manner one creates not only a precisely formed passage opening at the distal prosthesis shaft end which, endowed with the full stability of the connection of the circular lamellas with the shaft wall, is molded into the latter, but also, in particular, contact areas running parallel to the inside as well as the outside which provide ideal conditions for the attachment of connecting pieces and ulterior prosthesis elements. All connecting components thus remain exchangeable.

### BRIEF DESCRIPTION OF THE SEVERAL VIEWS OF THE DRAWINGS

Additional advantages and characteristics of the invention become evident in the following descriptions of examples of implementation.

FIG. 1 shows a shaft system according to the invention in a perspective view with a partial section through the passage opening.

FIG. 2 shows the distal shaft end with a passage opening in perspective view.

FIG. 3 shows a reinforcing element with a schematically shown course of fibers for the fiber-reinforced plastic of a prosthesis shaft.

FIG. 4 shows the section view of a reinforcing element with fibers surrounding it.

FIG. 5 shows a perspective view of reinforcing elements with borings corresponding to a four-hole plate.

FIG. 6 shows the distal end of a prosthesis shaft with a passage opening and a prosthesis connecting element inserted into it. FIG. 6A is an isolated view showing the fibers, end bead and reinforcement elements.

FIG. 7 shows a prosthesis shaft during its manufacture.

FIG. 8 shows a prosthesis shaft during a further step in its manufacture.

FIG. 9 shows an example of implementation of a reinforcing element with an extension.

FIG. 10 shows an example of implementation of a reinforcing element in a perspective view.

FIG. 11 shows a top view of a reinforcing element in a different implementation.

### DETAILED DESCRIPTION OF THE INVENTION

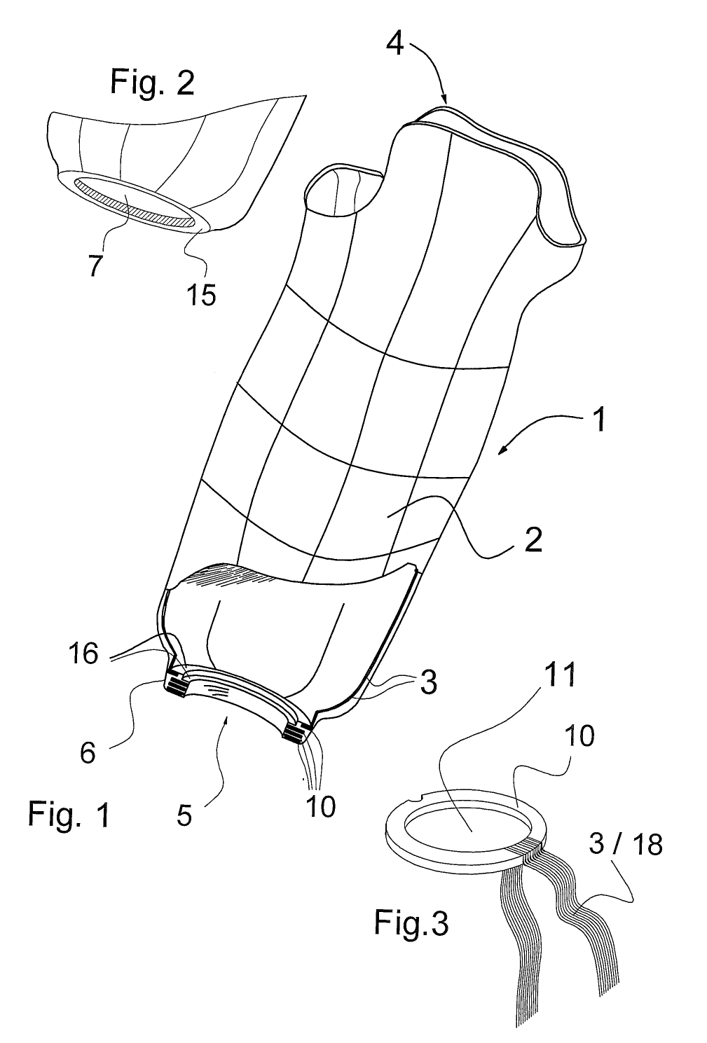

FIG. 1 shows a shaft system 1 for a prosthesis according to an example of implementation of the present invention. Visible is a prosthesis shaft for a stump of an upper or lower leg with a shaft wall 2 made of plastic material reinforced with fibers 3.

This prosthesis shaft has a proximal opening 4 for accepting an amputation stump and a distal end 5 with a passage opening 7. In the example shown here, this passage opening is essentially in the shape of a circle. Its shape may however also be oval, polygonal or angular.

At the distal end 5 one recognizes in a partial cutaway the schematically represented synthetic fibers 3 of the shaft wall 2 as they are wound around in several reinforcements 10 arranged around the passage opening 7 and thereby building up the latter to an end bead 6.

In this example of implementation four reinforcements 10 are integrated into the end bead 6. Ideally, to every layer of fibers 3 reinforcing the plastic material, a reinforcement 10 is attributed by wrapping the fiber fabric 3 around these reinforcements 10 and so envelops these reinforcements as completely as possible. This creates an inseparable connection between the fibers 3 reinforcing the plastic material of the prosthesis shaft 2 and the essentially superimposed reinforcements 10 that build up the end bead 6.

Depending on the requirements, the number of superimposed reinforcements 10 can be increased, the stability of the end bead 6 increasing in reproducible manner with each layer added in this way.

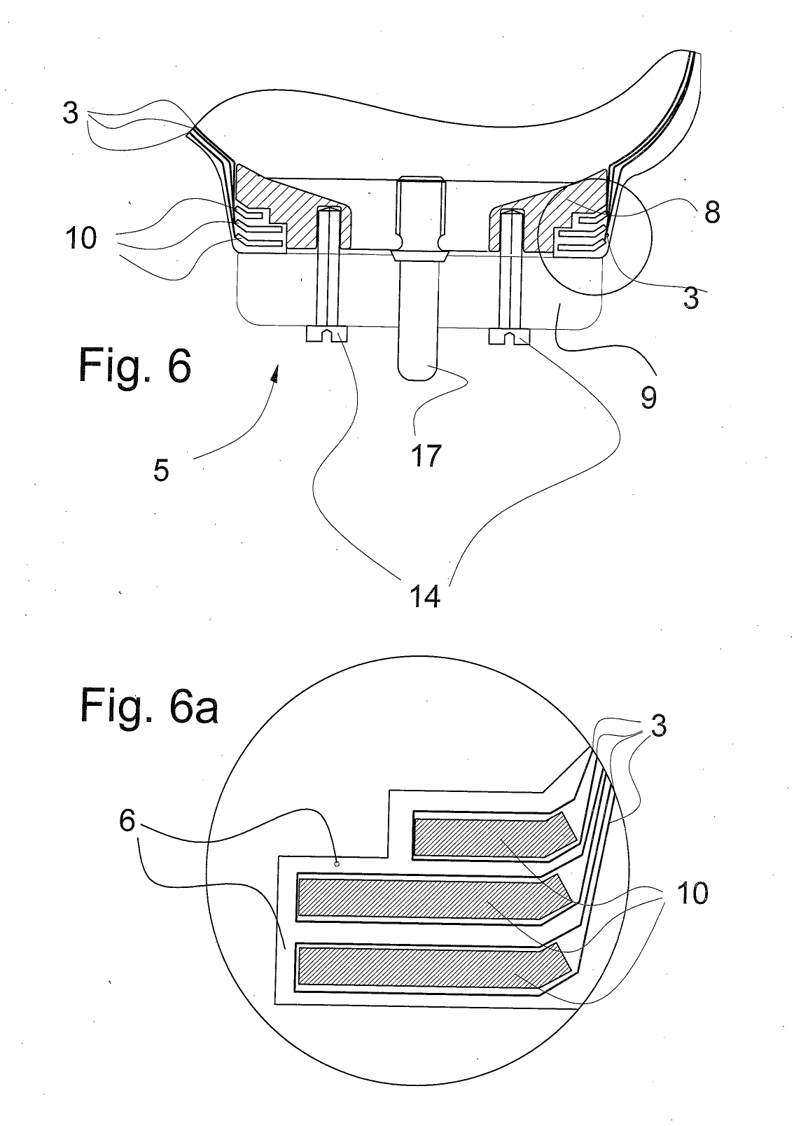

On the end bead 6, one can identify a bearing surface 16 which is situated on the inside of the prosthesis shaft and the contour of which is created in the manufacturing process described below, in order to reach a counterfort especially defined in its shape for a clamping plate 8 attached here for a connecting element 9, as shown in FIG. 6 and explained further below.

In the example of implementation shown here in FIG. 1 in particular, the passage opening 7 and the end bead 6 have a circular shape so that all connecting elements can be rotated into any desired position and can be arrested in relation to the prosthesis shaft 1.

All prostheses components to be coupled onto the prosthesis shaft can easily be attached and removed.

The layers of reinforcement elements 10 may consist of various materials. In a preferred implementation, they are made of metal to achieve a high degree of stability and a low overall height. Thereby it is also pos-sible to keep the overall height of the end bead 6 with an optimal connection to the shaft wall 2 made of fiber-reinforced synthetic material as low as possible.

FIG. 2 shows in a perspective view the distal end 5 of a prosthesis shaft 2, where on the outside of an end bead 6 a bearing surface 15 is visible, on which a prosthesis connecting element as shown in FIG. 6 is to be attached in the passage opening 7 at the end bead 6.

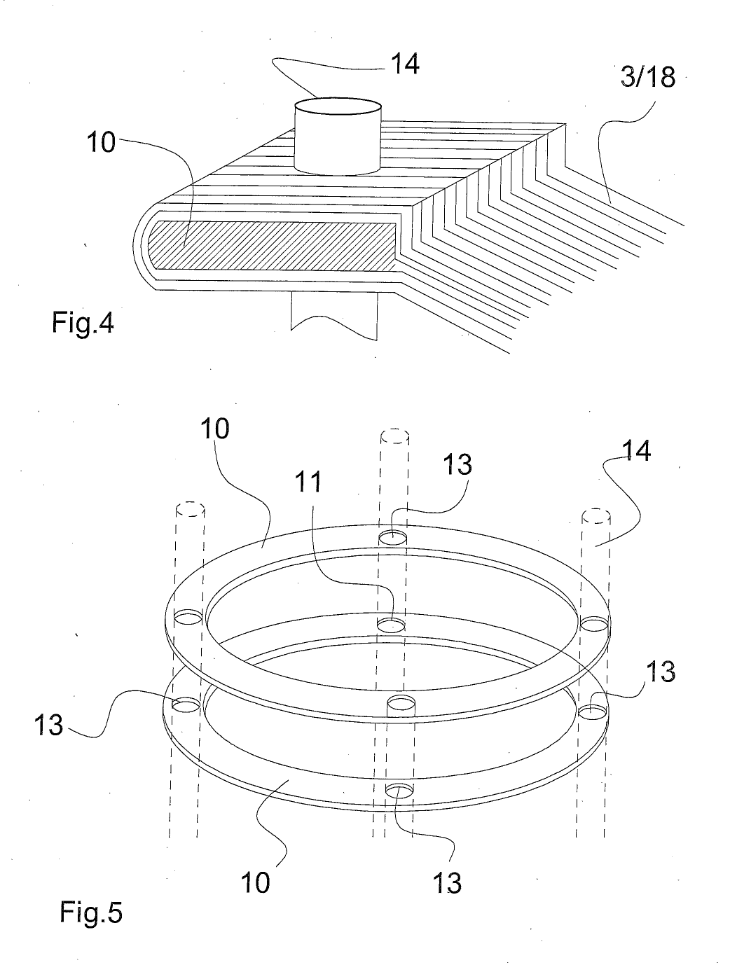

FIG. 3 shows in a perspective view a schematically presented reinforcement element 10 in the form of a circular lamella with a fiber 3 running uninterruptedly through the interior of the ring. A centrally located opening 11 provides sufficient room for this leadthrough of the entire fabric made of fibers 3 and a plastic matrix of the prosthesis shaft as well as for the leadthrough of functional parts 17, tensioning elements 14 and for the connection of prosthesis components 9, as shown in FIG. 6.

If the reinforcement element 10 is executed in metal, during the tie-off and the simultaneous heating of the fabric consisting of fibers 3, the reinforcement element 10 will also expand. During the subsequent cooling, the reinforcement element 10 will contract again and will wrap itself around the plastic material in circular and form-fitting fashion. The expansion and subsequent contraction of the metal are more pronounced than on the plastic material thereby strengthening the bond between the reinforcement element and the plastic material.

FIG. 4 shows a section through a reinforcement element 10 with the course of fibers 3 wrapped around a reinforcement element 10. One can see how the fibers 3 are tightly running over all sides of the reinforcement element 10 without any interruption. The close contact of the fibers 3 with the reinforcement element 10 is so pronounced that no additional conglutination of the fibers with the reinforcement element 10 is necessary.

In this example of implementation it is also possible to discern the installation of a tensioning element 14. With this tensioning element 14 it is possible, when necessary, to tension and keep together several layers of wrapped and stacked reinforcement elements 10.

For this purpose, FIG. 5 shows a preferred execution of reinforcement elements 10 which are, virtually like a known four-hole plate, equipped with a large central opening 11 through which, during the manufacture of the prosthesis shaft, the fibers 3 are passed for the purpose of strengthening of the plastic material constituting the latter.

The reinforcing elements are provided with holes 13. These are intended for the passage of tensioning elements 14 and may also be used for the connection of prosthesis components that are not shown here. These holes 13 are being kept open during the manufacturing process of the connection element using place-holders for such tensioning elements 14.

FIGS. 6 and 6A show a section through the distal end 5 of the shaft system.

One can see the end bead 6 with several superimposed reinforcement elements 10 and the schematically shown reinforcement with fibers 3 of the shaft wall which is installed in the end bead 6.

In this example of implementation, the end bead 6 represented here is provided on its exterior and on its interior side with steps and so offers an appropriate seat for a clamping plate 8 installed from the proximal direction, and from the distal direction a corresponding connection element 9 between which the end bead 6 and the reinforcement elements 10 inside the latter are braced by means of these connecting tension elements 14.

In this variant, not only the fibers 3 which reinforce the plastic material of the prosthesis shaft are installed through the opening 11 of the reinforcement elements 10, but also the tensioning elements 14 are passed through the opening passage 7 which is formed by the opening 11.

In the design version shown here the connecting element 9 which is adjustably connected with the clamping plate 8 via the tensioning elements 14 in the form of screws, also features a functional part 17, for example in the form of a liner lock shown here but only in schematic form.

The connecting element 9 is provided for the connection of prosthesis components which are not shown here. It becomes clear in this representation that only the reinforcing elements 10 enter into an inseparable connection with the shaft wall 2, whereas the connecting element 9 and consequently all prosthesis elements to be mounted on the prosthesis shaft via this connecting element 9 and thus utilized in this shaft system 1 have to be done in a detachable manner.

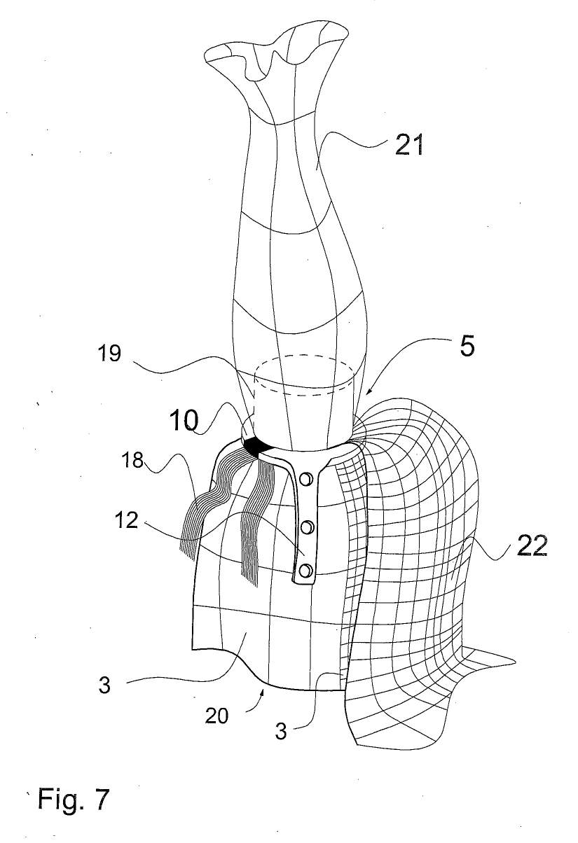

FIG. 7 shows in a perspective view the shaft system 1 while in production.

In a first manufacturing step a place-holder 19 is installed on a shape model 20 of an amputation stump, at the distal end of the future shaft.

The shape model 20 and the place-holder 19 are being covered with a hose fabric of fibers 3 while keeping an excess length 21. Over this excess length, a reinforcing element 10 in the form of a circular lamella is placed on the place-holder which is also called a “dummy”. This rein-forcing element 10 fixates the fabric hose of fibers 3 with its excess length 21 and possible also additional reinforcing strips 18 on the place-holder.

Also shown in FIG. 7 is an extension 12 which protrudes from the reinforcement element 10 in its form of a circular lamella. This extension 12 is placed on the shape model 20 and thus effects an additional stabilization of the end bead 6.

Later on, during the manufacturing process, the excess length 21 of the fiber fabric is draped over the reinforcing element 10 and back over the shape model 20, with the reinforcing element being completely enveloped by the fibers 3 of the fiber fabric, so that it will later be firmly connected without interruption of the plastic structure with the fiber-reinforced plastic material of the shaft wall.

This process is repeated as often as needed, so that any number of layers of fibers 3 can be bundled in the area of the end bead 6.

It is also possible to provide individual fabric strips 22 instead of a fabric hose, in order to achieve targeted reinforcement of the shaft wall.

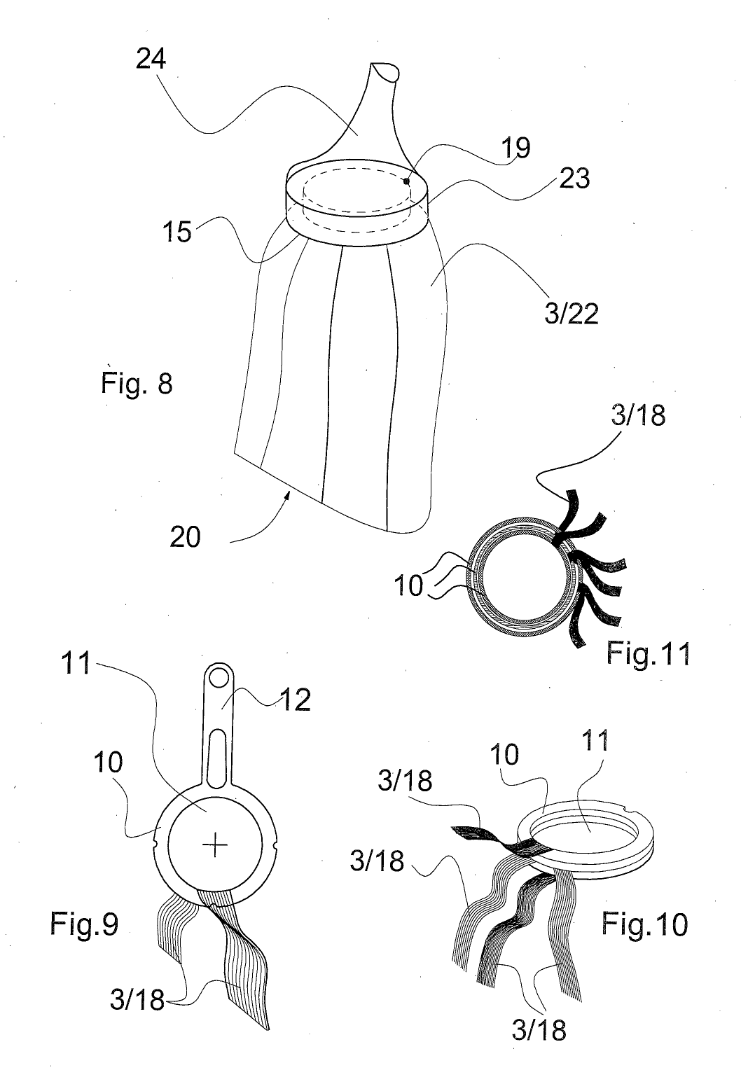

FIG. 8 shows in a perspective view the last manufacturing step in a schematic representation.

Here already several layers of fibers 3—which are no longer separately recognizable particularly if processed in fabric hoses—have been placed over several reinforcing elements 10 on the shape model 20. The superimposed fabric hoses form a homogeneous structure at the distal end 5 of the shaft, with the place-holder 19 being exposed and protruding from the shaft wall.

A structural part 23 is put over the place-holder 19 and is in contact with the fabric of fibers 3.

For the subsequent pouring of plastic material, a vacuum hose 24 is attached to the prepared, fabric-covered molded blank of the shaft.

During the subsequent casting process, due to the interior shape of the molded part 23 and the shape of the dummy and the place-holder at the end bead 6, several fully shaped bearing surfaces 15, 16 are created at the distal end 5 of the prosthesis shaft.

Excess casting resin is isolated from the prosthesis shaft by the molded part 23, thereby making unnecessary any finishing work such as for example grinding off the end bead in order to create plane bearing surfaces.

After the casting the molded part 23 and the place-holder 19 are removed.

The simple manufacturing method produces, without any finishing work, a fully formed end bead 6 around a passage opening 7 with precisely formed bearing surfaces 15, 16 for a form-fitting and highly stressable connection of the prosthesis shaft with attachable prosthesis components.

FIG. 9 shows a top view of a reinforcing element 10 with an extension 12 and the principle of the fibers 18 introduced through the central opening 11 of the reinforcement element 10 in the form of a circular lamella, thereby ensuring their intimate adhesion to the reinforcement element 10.

The reinforcement element 10 may also be equipped with several extensions 12. Essentially, the reinforcement elements 10 were inexpensively producible molded parts not intended for re-use and remaining in the prosthesis shaft. In especially preferred variants these parts are stamped or produced in an injection molding process.

FIG. 10 shows in a perspective view the schematic structure inside the end bead 6 with two, relative to the passage opening 7 which is sur-rounded by the end bead, essentially axially layered reinforcement elements 10, and the course of the fibers 3 passing between these. In a particularly effective implementation every individual layer of the fibers 3 which later strengthen the plastic material of the prosthesis shaft, is placed, together with additional strengthening strip or ribbons 18 around each reinforcement element 10.

FIG. 11 shows in a top view the schematic structure with a different arrangement inside the end bead 6 with radially superimposed reinforcement elements 10. These have increasingly larger free interior diameters so that the inner openings 11 can accept somewhat smaller reinforcement elements with fibers or strengthening strips wrapped around them. This method of construction is particularly appropriate when a lower height is required for an end bead.

In a particularly preferred implementation every individual layer of plastic-strengthening fibers 3 is placed with strengthening ribbons 18 around each reinforcement element 10.

### LIST OF COMPONENTS IN DRAWINGS

1. Shaft system
2. Shaft wall
3. Fibers
4. Proximal opening
5. Distal end
6. End bead
7. Passage opening
8. Clamping plate
9. Connecting piece
10. Reinforcements
11. Centrally located opening
12. Extension
13. Holes
14. Tensioning elements
15. Bearing surface
16. Bearing surface
17. Functional parts
18. Additional strengthening strips
19. Place-holder
20. Shape model
21. Overlap or excess
22. Fabric strip
23. Molded part

1. Shaft system for a prosthesis with the shaft system featuring a prosthesis shaft which has a shaft wall comprising plastic material reinforced with fibers and which features at its one end a proximal entrance opening and it its distal end a fastening system for the connection of prosthesis components and/or functional parts of the prosthesis such as locks, draw-in and connecting elements, with a passage opening at the distal end in the shaft wall of the prosthesis shaft, wherein the passage opening is provided with at least one reinforcement element surrounding it and wherein the fibers in the shaft wall are run through an opening in the middle of the reinforcing element.

2. Shaft system according to claim 1, wherein several reinforcement elements are present which are interconnected by means of tensioning elements.

3. Shaft system according to claim 1, wherein the at least one reinforcement element is in the form of a circular lamella and features at least one extension in radial direction which can be positioned between layers of fiber reinforcements for the plastic material of the shaft wall.

4. Shaft system according to claim 1, wherein the passage opening is provided with an end bead featuring at least one bearing surface.

5. Shaft system according to claim 1, wherein a portion of a prosthesis component and/or of a functional part of the prosthesis passing through the passage opening can be anchored on the wall of the prosthesis shaft in a rotable and arrestable manner in respect to the stump axis.

6. Method for the manufacture of a shaft system according to claim 1 with these steps:
a) Installation of a place-holder on a shape model of an amputation stump,
b) Enveloping the place-holder and the shape model with fibers for a fiber-reinforced plastic material in approximately twice its length with creation of an excess,
c) Passing the excess length through a centrally located opening of a reinforcement element and arranging the reinforcement element on the place-holder, as well as
d) Folding back the excess length on the reinforcement element and placement on the shape model and
e) Enclosing the shape model, the fibers and of the returned excess length with a vacuum hose and filling with a plastic material matrix.

7. Method for the manufacture of a shaft system according to claim 6, wherein the steps b) to d) are repeated several times prior to performing step e).

8. Method for the manufacture of a shaft system according to claim 6, wherein after step d) and before step e) there is a step during which a molded part is attached on the place-holder which determines the contour of the passage opening.
